(19) United States
(12) Patent Application Publication
(10) Pub. No.: US 2026/0144812 A1
(43) Pub. Date: May 28, 2026

(54) ANTIOXIDANT OPHTHALMIC EMULSION

(71) Applicant: Alcon Inc., Fribourg (CH)

(72) Inventors: Mohammad Mostafa Kamal, Irving, TX (US); Jianheng Wen, Duluth, GA (US); Ye Hong, Flower Mound, TX (US)

(21) Appl. No.: 19/400,442

(22) Filed: Nov. 25, 2025

Related U.S. Application Data
(60) Provisional application No. 63/726,076, filed on Nov. 27, 2024.

(51) Int. Cl. A61K 31/728; A61K 9/00; A61K 9/107; A61K 31/355; A61K 47/02; A61K 47/06; A61K 47/10; A61K 47/24; A61K 47/26; A61K 47/36

(52) U.S. Cl. CPC A61K 31/728; A61K 9/0048; A61K 9/1075; A61K 31/355; A61K 47/02; A61K 47/06; A61K 47/10; A61K 47/24; A61K 47/26; A61K 47/36

(57) ABSTRACT

The present invention is directed to an antioxidant ophthalmic emulsion. More particularly, the present invention is directed to an ophthalmic emulsion having a unique combination of ingredients that promotes the transparent property of small oil droplets within the emulsion and promotes the therapeutic delivery capability of the emulsion.

### TECHNICAL FIELD OF THE INVENTION

The present invention is directed to an antioxidant ophthalmic emulsion. More particularly, the present invention is directed to an ophthalmic emulsion having a unique combination of ingredients that promotes the transparent property of small oil droplets within the emulsion and promotes the therapeutic delivery capability of the emulsion.

### BACKGROUND OF THE INVENTION

There are a variety of types of ophthalmic compositions such as aqueous solutions, aqueous suspensions and emulsions.

Ophthalmic aqueous solution compositions for topical application, and in particular artificial tear compositions, comprise compounds that lubricate and protect the ocular surface. In the context of dry eye disorders, artificial tear compositions can prevent symptoms such as pain and discomfort and can prevent bioadhesion and tissue damage induced by friction. A large number of potential compounds are available that are useful as lubricants and ocular surface protectants. For example, certain marketed artificial tear products contain natural polymers such as galactomannans. Other lubricants and ocular surface protectants include, for example, carboxymethylcellulose, glucomannan, glycerol, and hydroxypropylmethylcellulose. As noted above, ophthalmic compositions have been previously described that utilize galactomannan compounds such as guar. U.S. Pat. No. 6,403,609 to Asgharian, entitled “Ophthalmic compositions containing galactomannan polymers and borate,” describes such systems and is herein incorporated by reference in its entirety.

Hyaluronic acid (HA) occurs naturally in the human body and Sodium hyaluronate is the water-soluble salt form of hyaluronic acid, a biocompatible polysaccharide with unique hygroscopic and viscoelastic properties. as a natural lubricant and its water-retaining properties make it well-suited for the ocular surface to help provide enhanced hydration and comfort to dry eye patients. Sodium hyaluronate has been shown to effectively treat symptoms of dry eye. Sodium hyaluronate eye drops increase precorneal tear film stability and corneal wettability, reduce the tear evaporation rate, and the healing time of corneal epithelium (Aragona P, Di Stefano G, Ferreri F, et al. Sodium hyaluronate eye drops of different osmolarity for the treatment of dry eye in Sjogren's syndrome patients. Br J Ophthalmol 2002 August; 86 (8): 879-84).

The combination of sodium hyaluronate and hydroxypropyl guar in a single aqueous solution formulation provided an effective moisture layer that resulted in significantly greater cell viability after desiccation than either polymer alone, and protection from desiccation was evident even after test solutions were rinsed away. U.S. Pat. No. 10,828,320 to Davis et. Al. entitled “Ophthalmic Compositions With Improved Dessication Protection And Retention” describes such systems and is herein incorporated by reference in its entirety.

Contrast to above ophthalmic aqueous solution compositions, ophthalmic compositions are also formulated as emulsions. Ophthalmic emulsions are typically employed in circumstances where it is desirable to include two or more ingredients that are immiscible relative to each other in a single composition and therefore form two separate phases within the composition. Such emulsions can allow a single composition to provide the advantages attributable to both phases (e.g., advantageous delivery characteristics). For example, an emulsion can be formed of oil droplets in an aqueous phase where the oil droplets can be used as carriers for actives such as therapeutic agents (e.g., drugs) or excipients which have poor solubility and/or stability in water. Examples of emulsions are included in U.S. Pat. Nos. 4,914,088; 5,278,151; 5,294,607; 5,371,108; and 5,578,586. Each of these patents is incorporated herein by reference for all purposes.

It is typically quite desirable for one phase of an emulsion to be substantially uniformly dispersed within the other phase. Such dispersion can significantly effect the capabilities of emulsion to deliver therapeutic ingredients. Moreover, such dispersion is often an indication of the stability of the emulsion itself.

The separate phases of an emulsion can be extremely difficult to evenly disperse throughout a composition since each phase tends to associate with itself rather than the other phase. Thus, the maintenance of the distribution of one phase (i.e., the dispersed phase) within the other phase (i.e., the continuous phase) can be very delicate. Moreover, it is also often difficult to include additional ingredients within an emulsion since many ingredients can act to inhibit the dispersion and/or even distribution of the dispersed phase throughout the continuous phase.

The ophthalmic emulsion compositions have been previously described that utilize galactomannan compounds such as guar. U.S. Pat. No. 10,004,685 to Ketelson entitled “Ophthalmic Emulsion,” describes such emulsion systems and is herein incorporated by reference in its entirety. However, it is difficult to incorporate water soluble polymers such as sodium hyaluronate into ophthalmic oil-in-water emulsions.

In addition, oxidative eye damage is a result of an imbalance between the production of reactive oxygen species (ROS) and the body's ability to fight them off with antioxidants. This imbalance can lead to oxidative stress, which can damage the eye's cells and contribute to a number of eye diseases. Topical application of antioxidant on the eye may neutralize or remove free radicals by donating an electron. The neutralizing effect of antioxidants helps protect the eye from oxidative stress harmful free radicals. Examples of antioxidants include vitamins A, C, and E. Like free radicals, antioxidants come from several different sources.

Accordingly, there is a need for an antioxidant ophthalmic emulsion containing an antioxidant, galactomannan compounds and sodium hyaluronate polymer and ophthalmic emulsion is optically transparent and the ophthalmic emulsion eye drops coat and protect the surface of the eye, while providing hydration and lubrication.

### SUMMARY OF THE INVENTION

The present invention is directed to an antioxidant containing ophthalmic emulsion, the emulsion comprising:
water forming an aqueous phase;
oil forming an oil phase, wherein oil in amount from 0.8 to 1.2 w/v %;
a charged phospholipid;
a first surfactant, wherein the first surfactant is a vitamin-based surfactant (vitamin E TPGS),
a second surfactant, the second surfactant is selected from a group of surfactants consisting of polyoxyethylene sorbitan monooleate (Tween 80), and a hydrophilic surfactant having an HLB value having an HLB value of from 10 to 18, wherein the hydrophilic surfactant does not include vitamin E TPGS and polyoxyethylene sorbitan monooleate, and a hydrophobic surfactant having an HLB value of from 1 to 6, and a combination thereof; borate;
   two mucoadhesive polymers, the first mucoadhesive polymer is sodium hyaluronate, the second mucoadhesive polymer is galactomannan polymer;
wherein a ratio of the total concentration of the two mucoadhesive polymers to the total concentration of the first surfactant and the second surfactant is between 0.20 and 0.40;
wherein a ratio oil to the total concentration of the first surfactant and the second surfactant is between 0.7 and 1.1;
wherein the ophthalmic emulsion is transparent;

The borate and galactomannan polymer cooperatively act to form a gel upon instillation of the emulsion in an eye of an individual.

### DETAILED DESCRIPTION OF THE INVENTION

The present invention is predicated upon the provision of an ophthalmic oil in water emulsion wherein the emulsion has average oil droplet size that is relatively small. The emulsion will typically be aqueous and include a substantial amount of water. The emulsion will also typically include an anionic phospholipid, a hydrophilic surfactant (high HLB) and a hydrophobic (low HLB) surfactant. Further, the emulsion will typically include two mucoadhesive ingredients (e.g., galactomannan polymers and sodium hyaluronate) to aid in maintaining the emulsion on the corneal surface of the eye and/or aid in delivering one or more lipophilic compounds to the corneal surface. The emulsions of the present invention are most desirably used for dry eye therapeutics. However, without limitation, it is also contemplated that the emulsions may be used for drug delivery, vitamin delivery, botanical delivery, contact lens wetting and contact lens lubrication.

Unless otherwise specifically stated all emulsion ingredient amounts or percentages are weight volume percentages (w/v %).

The present invention is partly based on the finding that vitamin E TPGS not only uses as alternatives to vitamin E but also as an effective emulsifier for the emulsion. Vitamin E has been identified as an essential factor for reproduction since 1922. With further investigation, it has been found with other functions involving antioxidant, anti-thrombolytic and other therapeutic effects. However, the poor water solubility of vitamin E has greatly limited its application. Vitamin E d-α-tocopheryl poly(ethylene glycol) 1000 succinate (simply as Vitamin E TPGS or TPGS), synthesized by esterification of vitamin E succinate with poly(ethylene glycol) (PEG) 1000, is a water-soluble derivative of natural vitamin E. It has an amphiphilic structure comprising hydrophilic polar head portion and lipophilic alkyl tail. TPGS can be functionalized as an excellent solubilizer, emulsifier.

The present invention is also partly based on the finding that if the vitamin E TPGS containing emulsion of the present invention has an emulsion having a ratio oil to the total concentration of two surfactants is between 0.70 and 1.2, preferably between 0.80 and 1.10, more preferably between 0.85 and 1.00 and a ratio of the total concentration of the two mucoadhesive polymers to the total concentration of two surfactants is between 0.20 and 0.40, preferably between 0.25 and 0.35, more preferably between 0.26 and 0.3, the ophthalmic emulsion is transparent. preferably between 0.80 and 1.10

Visually Opaque emulsions with larger droplet sizes often causes transient vision blurring upon instillation. It also reduces consumer preferences compared to transparent competing products. Additionally, there is a need of antioxidant enriched lubricating eye drops to reduces oxidative stress in lachrymal fluid. Antioxidants reduces oxidative stress in lachrymal fluid and enhances the protection of corneal and conjunctival epithelial cells. Invented optically transparent emulsions include existing Systane complete platform or novel emulsion platform with additional emulsifier/excipients. The present inventive emulsion integrates antioxidant materials Vitamin E to provide with additional benefit for ocular health. The transparent emulsion will not only generate superior patient perception but will also be compatible with transparent packaging system/requirements. The reduced particle size in the transparent emulsion will also reduce vision blurring upon instillation and enhanced physical stability of the product

Unless otherwise specifically stated all emulsion ingredient amounts or percentages are weight volume percentages (w/v %).

The oil of the emulsion is dispersed throughout the continuous water or aqueous phase as small droplets that are substantially distinct and separate. The oil phase is in droplets within the aqueous phase and the droplets have a D90 diameter that is no greater than 120 nm but is at least 50 nm; determined using Anton Paar Litesizer 500 DLS as the droplet diameter corresponding to 90% of the cumulative undersize distribution by volume”

Particle size analyzers may be used to determine emulsion oil droplet size. For example, Anton Paar Litesizer 500 is a dynamic light scattering (DLS) device that can be used to measure the emulsion oil droplet size and droplet size distribution. DLS derives particle size information from the time dependence of the scattered laser radiation intensity that passes through the emulsion. DLS software optimizes the measurement conditions, such as the detection angle, and uses the time dependence of the scattered intensity (autocorrelation function) to calculate the average size of the particles (hydrodynamic radius) as well as the size distribution. The droplet diameter corresponding to 90% of the cumulative undersize distribution by intensity is used.”

The emulsion of the present invention is an oil in water emulsion. The oil can be any of numerous mineral, vegetable, and synthetic substances and/or animal and vegetable fats or any combination of oils. The oil can be soluble in various organic solvents such as ether but not in water. The oil phase can comprise, if desired a liquid hydrocarbon, such as a mineral oil, paraffin oils, petrolatum or hydrocarbon oils. Mineral oil is particularly preferred. A silicone oil may also be used. The oil phase can additionally include a waxy hydrocarbon, such as paraffin waxes, hydrogenated castor oil, Synchrowax HRC, Carnabau, beeswax, modified beeswaxes, microcrystalline waxes, and polyethylene waxes.

The oil is typically at least 0.01 w/v %, more typically at least 0.1 w/v % and even more typically 0.8 w/v % of the emulsion. The oil is also typically no greater than about 20 w/v %, more typically no greater than about 5 w/v % and even more typically no greater than about 3 or even no greater than 1.5 w/v % of the emulsion. The oil is typical present in an amount of in amount from 0.8 to 1.2 w/v %, preferably between about 0.9 and about 1.15 wt/v %, more preferably between about 0.9 and about 1.1 wt/v %, most preferably 1 wt/v %.

According to the present invention, the antioxidant emulsion comprises vitamin E tocopheryl derivatives which are water-soluble, biologically-active vitamin E analogues. These vitamin E derivatives have been used as alternatives to vitamin E, especially where water-solubility is desired. Vitamin E derivatives in the present invention provide not only an antioxidant property but also a surfactant property.

Vitamin E TPGS has an array of applications in Cosmetic and Personal Care formulations. Not only has it been widely used in drugs to improve absorption and bioavailability, but it also works as an emulsifier/co-emulsifier for lipophilic actives or ingredients while simultaneously providing a water-soluble source of natural vitamin E. The science of vitamin E TPGS has been extensively studied and reported worldwide, showing/proving it to be one of the best and safest options for emulsifiers in skincare and other personal care products.

TPGS is synthesized from the lipid-soluble antioxidant, α-tocopherol (vitamin E) by grafting to a polyethylene glycol (PEG) oligomer through a succinate diester linker.

Vitamin E tocopheryl derivatives are water-soluble, biologically-active vitamin E analogues. These vitamin E derivatives have been used as alternatives to vitamin E, especially where water-solubility is desired.

The vitamin E tocopheryl derivatives useful in the compositions of the present invention are highly water-soluble polyoxyalkylene glycol esters of vitamin E tocopheryl esters of a dicarboxylic acid. Representative esters of this type include the polyoxyethylene glycol esters of vitamin E tocopheryl esters of a dicarboxylic acid wherein the polyoxyethylene glycol moiety of the ester (sometimes merely referred to as the polyoxyethylene glycol moiety of the ester) has a molecular weight in the range from about 600 to about 6000, preferably in the range from about 600 to about 1500. Such esters and methods for their preparation are disclosed in U.S. Pat. No. 2,680,749 (Cawley et al.). The most preferred ester is the .alpha.-tocopheryl polyoxyethylene glycol (1000) succinate, a polyoxyethylene glycol ester of .alpha.-tocopheryl succinate wherein the polyoxyethylene glycol moiety of the molecule has an average molecular weight of about 1000.

Vitamin E Tocopherol Polyethylene Glycol 1000 Succinate (Vitamin ETPGS) is an amphipathic excipient which is a water soluble derivative of natural-source vitamin E. Vitamin ETPGS, or PEGylated vitamin E, is a vitamin E derivative in which polyethylene glycol subunits are attached by a succinic acid diester at the ring hydroxyl of the vitamin E molecule. Vitamin ETPGS is a hydrophilic non-ionic surfactant with an HLB index of about 13.

In general, one or more vitamin E derivatives are used in the compositions of the present invention in an amount between about 0.1 and about 5 wt/V %, preferably between about 0.3 and about 3 wt/v %, more preferably between about 0.4 and about 2 wt/v %, most preferably between about 0.5 and about 1 wt/v %.

In addition to vitamin-based surfactant (vitamin E TPGS), the emulsion of the present invention may contain a second surfactant, and the second surfactant is selected from a group of surfactants consisting of polyoxyethylene sorbitan monooleate (Tween 80), a third surfactant, wherein the third surfactant is hydrophilic surfactant having an HLB value having an HLB value of from 10 to 18 (or) great than 16, a fourth surfactant, wherein the fourth surfactant is a hydrophobic surfactant having an HLB value of from 1 to 6, and a combination thereof, wherein the third surfactant is not polyoxyethylene sorbitan monooleate.

Tween 80 is also a polyethylene sorbitol ester, otherwise known as Polysorbate 80 or polyoxyethylene sorbitan monooleate. It has a molecular weight of 1.31 kDa and works well as a stabilizer and emulsifier. Tween is a polysorbate nonionic surfactant used in many drug product formulations including parenterals and certain vaccines. They consist primarily of fatty acid esters of polyethoxy sorbitan. Tween 80® (also known as polysorbate 80 is formulated to contain the ethoxylated oligomers with different fatty acid side chains, ethoxylated sugars and PEG oligomers. The nonionic detergents Tween 80 (polyoxyethylene sorbitan monooleate).

In the compositions of the present invention, Tween 80 is present in an amount between about 0.1 and about 2 wt/V %, preferably between about 0.3 and about 1 wt/v %, more preferably between about 0.5 and about 0.6 wt/v %, even more preferably about 0.5 wt/v %.

The hydrophilic surfactant can be a fatty acid, an ester, an ether, an acid or any combination thereof. The hydrophilic surfactant may be ionic or non-ionic, but is preferably non-ionic. Many suitable surfactants/emulsifiers are nonionic ester or ether emulsifiers comprising a polyoxyalkylene moiety, especially a polyoxyethylene moiety, often containing from about 2 to 80, and especially 5 to 60 oxyethylene units, and/or contain a polyhydroxy compound such as glycerol or sorbitol or other alditols as hydrophilic moiety. The hydrophilic moiety can contain polyoxypropylene. The emulsifiers additionally contain a hydrophobic alkyl, alkenyl or aralkyl moiety, normally containing from about 8 to 50 carbons and particularly from 10 to 30 carbons. Examples of hydrophilic surfactants/emulsifiers include ceteareth-10 to-25, ceteth-10-25, steareth-10-25, and PEG-15-25 stearate or distearate. Other suitable examples include C10-C20 fatty acid mono, di or tri-glycerides. Further examples include C18-C22 fatty alcohol ethers of polyethylene oxides (8 to 12 EO). One particularly preferred hydrophilic surfactant is polyoxyethylene-40-stearate, which is sold under the tradename MYRJ-52, which is commercially available from Nikko Chemicals.

The hydrophilic surfactant is typically present in the emulsion in an amount that is at least about 0.01 w/v %, more typically at least about 0.08 w/v % and even more typically at least about 0.15 w/v %. The hydrophilic surfactant is typically present in the emulsion in an amount that is no greater than about 1.0 w/v %, more typically no greater than about 0.8 w/v % and even more typically no greater than about 0.4 w/v %. The hydrophilic surfactant is typically present in the emulsion in an amount between 0.15 w/v % to 1.0 w/v %, more typically between 0.25 w/v % to 0.8 w/v %, even more typically between 0.25 w/v % to 0.4 w/v %, still even typically 0.38 w/v %. According to the present invention, The hydrophilic surfactant does not include Tween 80 (polyoxyethylene sorbitan monooleate) and the charged phospholipid.

The hydrophobic surfactant can be a fatty acid, an ester, an ether, an acid or any combination thereof. The hydrophobic surfactant may be ionic or non-ionic, but is preferably non-ionic. The hydrophobic surfactant will typically include a hydrophobic moiety. The hydrophobic moiety can be either linear or branched and is often saturated, though it can be unsaturated, and is optionally fluorinated. The hydrophobic moiety can comprise a mixture of chain lengths, for example those deriving from tallow, lard, palm oil sunflower seed oil or soya bean oil. Such non-ionic surfactants can also be derived from a polyhydroxy compound such as glycerol or sorbitol or other alditols. Examples of hydrophobic surfactants include, without limitation, sorbitan fatty acid esters such as sorbitan monoleate, sorbitan monostearate, sorbitan monolaurate, sorbitan monopalmitate, sorbitan monoisostearate, sorbitan trioleate, sorbitan tristearate, sorbitan sesquioleate, sorbitan sesquistearate, combinations thereof or the like. One particularly preferred hydrophobic surfactant is a sorbitan tristearate sold under the tradename SPAN-65, which is commercially available from Croda Worldwide.

The hydrophobic surfactant is typically present in the emulsion in an amount that is at least about 0.01 w/v %, more typically at least about 0.08 w/v % and even more typically at least about 0.15 w/v %. The hydrophobic surfactant is typically present in the emulsion in an amount that is no greater than about 1.0 w/v %, more typically no greater than about 0.8 w/v % and even more typically no greater than about 0.4 w/v %. The hydrophobic surfactant is typically present in the emulsion in an amount between 0.15 w/v % to 1.0 w/v %, more typically between 0.25 w/v % to 0.8 w/v %, even more typically between 0.25 w/v % to 0.4 w/v %, still even typically 0.29 w/v %.

The types of galactomannans that may be used in the present invention are typically derived from guar gum, locust bean gum and tara gum. As used herein, the term “galactomannan” refers to polysaccharides derived from the above natural gums or similar natural or synthetic gums containing mannose or galactose moieties, or both groups, as the main structural components. Preferred galactomannans of the present invention are made up of linear chains of (1-4)-.beta.-D-mannopyranosyl units with .alpha.-D-galactopyranosyl units attached by (1-6) linkages. With the preferred galactomannans, the ratio of D-galactose to D-mannose varies, but generally will be from about 1:2 to 1:4. Galactomannans having a D-galactose:D-mannose ratio of about 1:2 are most preferred. Additionally, other chemically modified variations of the polysaccharides are also included in the “galactomannan” definition. For example, hydroxyethyl, hydroxypropyl and carboxymethylhydroxypropyl substitutions may be made to the galactomannans of the present invention. Non-ionic variations to the galactomannans, such as those containing alkoxy and alkyl(C1-C6) groups are particularly preferred when a soft gel is desired (e.g., hydroxylpropyl substitutions). Substitutions in the non-cis hydroxyl positions are most preferred. An example of non-ionic substitution of a galactomannan of the present invention is hydroxypropyl guar, with a molar substitution of about 0.4. Anionic substitutions may also be made to the galactomannans. Anionic substitution is particularly preferred when strongly responsive gels are desired. A galactomannan is typically present in a formulation of the present invention at a concentration of at least about 0.005 w/v %, more typically at least about 0.01 w/v % and even more typically at least about 0.03 w/v %, but typically no greater than about 5 w/v %, more typically no greater than about 1.0 w/v %, still more typically no greater than about 0.3 w/v % and even still more typically no greater than about 0.08 w/v %. The galactomannans is typically present in the emulsion in an amount between about 0.1 to about 0.2 w/v %, and more preferably between about 0.13 to 0.17 w/v %. In one embodiment, sodium hyaluronate is present at a concentration of about 0.15 w/v %.

Preferred galactomannans of the present invention are guar and hydroxypropyl guar.

Glycosaminoglycans such as hyaluronic acid are negatively charged molecules. Hyaluronic acid is an unsulphated glycosaminoglycan composed of repeating disaccharide units of N-acetylglucosamine (GIcNAc) and glucuronic acid (GIcUA) linked together by alternating beta-1,4 and beta-1,3 glycosidic bonds. Hyaluronic acid is also known as hyaluronan, hyaluronate, or HA. As used herein, the term hyaluronic acid also includes salt forms of hyaluronic acid such as sodium hyaluronate. Compositions of the present invention comprise from about 0.05 to about 0.5 w/v % hyaluronic acid. In a preferred embodiment, hyaluronic acid is present at a concentration of about 0.1 to about 0.2 w/v %, and more preferably at a concentration of about 0.13 to 0.17 w/v %. In one embodiment, sodium hyaluronate is present at a concentration of about 0.15 w/v %.

A preferred hyaluronic acid is sodium hyaluronate. The molecular weight of the hyaluronic acid used in compositions of the present invention may vary, but is typically 0.5 to 2.0 M Daltons. In one embodiment, the hyaluronic acid has a molecular weight of 900,000 to 1M Daltons. In another embodiment, the hyaluronic acid has a molecular weight of 1.9 to 2.0 M Daltons.

The emulsion may include additional or alternative polymeric ingredients and/or viscosity agents. Examples include, without limitation, carboxymethylcellulose, hydroxypropylmethyl cellulose, hydroxyethyl cellulose, carboxyvinyl polymer, xanthan gum, hyaluronic acid, any combinations thereof or the like.

The emulsion of the present invention includes at least one phospholipid for aiding in maintaining the stability of the emulsion and for reducing droplet size of the oil. It is known that complex phospholipids can contain a polar group at one end of their molecular structure and a non-polar group at the opposite end of their molecular structure. A discussion of phospholipids can be found in Lehninger, Biochemistry, 2 ed., Worth Publishers, New York, pp. 279-306, incorporated herein by reference for all purposes.

Many complex phospholipids are known to the art. They differ in size, shape and the electric charge of their polar head groups. Phosphoglycerides are compounds where one primary hydroxyl group of glycerol is esterified to phosphoric acid, and the other two hydroxyl groups are esterified with fatty acids. The parent compound of the series is, therefore, the phosphoric acid ester of glycerol. This compound has an asymmetric carbon atom and, therefore, the term phosphoglycerides includes stereoisomers. All phosphoglycerides have a negative charge at the phosphate group at pH 7, and the pKa of this group is in the range of 1 to 2. The head groups of phosphatidylinositol, phosphatidylglycerol including diphosphatidylglycerols (having the common name cardiolipins) and the phosphatidylsugars have no electric charge, and all are polar because of their high hydroxyl group content. Because of the negative charge of the phosphate group and the absence of a charge in the head group, the net charge of each of these materials is negative, and these materials are within the scope of the invention. Suitable phospholipids are those carrying a net positive or negative charge under conditions of use. The preferred materials are those carrying a net negative charge because the negatively charged material will be repelled by the negatively charged ocular surface thereby permitting the maintenance of a relatively thick aqueous layer upon application to the eye. The most preferred phospholipid is an anionic phospholipid named dimyristoyl phosphatidylglycerol (DMPG), which is a polyol with a net negative charge. Phosphatidylglycerol or a phosphatidylinositol are other examples. Suitable phospholipid additives are disclosed in the above cited U.S. Pat. No. 4,914,088, which is fully incorporated herein by reference for all purposes.

Most phospholipids are water insoluble. However, for application to the eye, it is desirable that the phospholipid be homogeneously distributed throughout an aqueous medium. For those few phospholipids having a solubility within a useful concentration range for use as a treatment composition, a simple aqueous solution of the phospholipid in saline is satisfactory. For those phospholipids that are essentially water insoluble, an aqueous composition in the form of an emulsion may be used. An emulsion provides a treatment composition where the phase containing the phospholipid component is homogeneously distributed throughout the aqueous vehicle.

The concentration of the phospholipid in the treatment composition may vary within wide limits. A treatment composition containing the complex phospholipid in an amount as low as 0.01 weight percent provides some benefit. When the treatment composition is in the form of an emulsion, compositions containing the phospholipid in elevated concentrations approaching collapse of the emulsion into separate aqueous and phospholipid phases is possible. A clinically practical concentration range for the phospholipid in its vehicle varies from about 0.05 to 7.0 w/v % phospholipid by weight, and more preferably varies from about 0.05 and 5.0 w/v %. In some embodiment of the present application, emulsion contains 0.005 w/v % phospholipid. It should be noted that the most desired concentration for the phosphor lipid in the aqueous composition will vary from subject to subject.

Other additives may be present in the phospholipid treatment composition including neutral lipids such as one or more triglycerides, cholesterol esters, the natural waxes and cholesterol; higher molecular weight isoprenoids; stabilizers; preservatives; pH adjustors to provide a composition preferably having a pH between about 6 and 8 and more preferably between about 7.0 and 7.4; salt in sufficient concentration to form an isotonic composition; medicants; etc.

As indicated above, the emulsions of the present invention can include borate or borate/polyol buffer systems. As used herein, the term “borate” includes boric acid, salts of boric acid, other pharmaceutically acceptable borates, and combinations thereof. The following borates are particularly preferred: boric acid, sodium borate, potassium borate, calcium borate, magnesium borate, manganese borate, and other such borate salts.

As used herein, the term “polyol” includes any compound having at least one hydroxyl group on each of two adjacent carbon atoms that are not in trans configuration relative to each other. The polyols can be linear or cyclic, substituted or unsubstituted, or mixtures thereof, so long as the resultant complex is water soluble and pharmaceutically acceptable. Examples of such compounds include: sugars, sugar alcohols, sugar acids and uronic acids. Preferred polyols are sugars, sugar alcohols and sugar acids, including, but not limited to: mannitol, glycerin, xylitol and sorbitol. Especially preferred polyols are mannitol and sorbitol; most preferred is sorbitol.

The use of borate-polyol complexes in ophthalmic compositions is described in U.S. Pat. No. 6,503,497 (Chowhan); the entire contents of which are hereby incorporated in the present specification by reference. The emulsions of the present invention preferably contain one or more borates in a concentration that is at least about 0.01% w/v, more typically at least about 0.3% w/v and even more typically at least about 0.8% w/v, but typically no greater than about 5.0% w/v, more typically no greater than about 2.0% w/v and even more typically no greater than about 1.2% w/v. It is generally desirable for the amount of the one or more borates to be sufficient to allow the formation of borate/polyol complexes and, when desired, to aid in gelling the galactomannan polymer upon application of the emulsion to the eye.

The compositions of the present invention may not include a preservative. However, the compositions of the present invention may include a preservative. Potential preservatives include, without limitation, hydrogen peroxide, chlorine containing preservatives such as benzalkonium chloride or others. According to a preferred aspect, however, the ophthalmic composition of the present invention is substantially free of any chloride containing preservatives and, particularly, is substantially free of benzalkonium chloride. Benzalkonium chloride, as a mixture of alkyl dimethyl benzyl ammonium having various alkyl chain lengths is used as preservative agent in topical ophthalmic products. Benzalkonium chloride also has cationic agent properties, and was used as cationic agents for emulsions, especially ophthalmic emulsions. According to the present, the ophthalmic emulsion composition do not contain benzalkonium chloride preservatives and benzalkonium chloride emulsion agent (surfactant).

Most preferred preservatives included in the ophthalmic composition are polymeric quaternary ammonium compounds. Still another most preferred option, no preservatives are included in the ophthalmic composition.

As used herein, the phrase “substantially free of” as it refers to an ingredient of the ophthalmic composition means that it is contemplated that the ophthalmic solution can be either entirely devoid of that particular ingredient or includes only a nominal amount of that particular ingredient.

The polymeric quaternary ammonium compounds useful in the compositions of the present invention are those which have an antimicrobial effect and which are ophthalmically acceptable. Preferred compounds of this type are described in U.S. Pat. Nos. 3,931,319; 4,027,020; 4,407,791; 4,525,346; 4,836,986; 5,037,647 and 5,300,287; and PCT application WO 91/09523 (Dziabo et al.). The most preferred polymeric ammonium compound is polyquaternium 1, otherwise known as POLYQUAD® or ONAMERM® with a number average molecular weight between 2,000 to 30,000. Preferably, the number average molecular weight is between 3,000 to 14,000.

The polymeric quaternary ammonium compounds are generally used in the compositions of the present invention in an amount that is greater than about 0.00001 w/v %, more typically greater than about 0.0003 w/v % and even more typically greater than about 0.0007 w/v % of the ophthalmic composition. Moreover, the polymeric quaternary ammonium compounds are generally used in the compositions of the present invention in an amount that is less than about 3 w/v %, more typically less than about 0.003 w/v % and even more typically less than about 0.0015 w/v % of the ophthalmic composition.

The emulsion of the present invention can include any of a multitude of ophthalmic therapeutic agents. Non-limiting examples of potential ophthalmic therapeutic agents for the present invention include: anti-glaucoma agents, anti-angiogenesis agents; anti-infective agents; anti-inflammatory agents; growth factors; immunosuppressant agents; and anti-allergic agents. Anti-glaucoma agents include beta-blockers, such as betaxolol and levobetaxolol; carbonic anhydrase inhibitors, such as brinzolamide and dorzolamide; prostaglandins, such as travoprost, bimatoprost, and latanoprost; seretonergics; muscarinics; dopaminergic agonists. Anti-angiogenesis agents include anecortave acetate (RETAANE™, Alcon™ Laboratories, Inc. of Fort Worth, Tex.) and receptor tyrosine kinase inhibitors (RTKi). Anti-inflammatory agents include non-steroidal and steroidal anti-inflammatory agents, such as triamcinolone actinide, suprofen, diclofenac, ketorolac, nepafenac, rimexolone, and tetrahydrocortisol. Growth factors include EGF or VEGF. Anti-allergic agents include olopatadine and epinastine. The ophthalmic drug may be present in the form of a pharmaceutically acceptable salt.

The present invention can be particularly useful for delivery therapeutic agents that relieve symptoms of dry eye conditions. Examples include, without limitation, steroidal and/or non-steroidal anti-inflammatory agents; selective PDE IV inhibitors such as cilomilast, cyclosporins, combinations thereof or the like. The emulsion of the invention can also be used in other fields, such as to deliver cooling agents, deliver antioxidants (omega-3 and omega-6 fatty acids) and other bioactivies for ophthalmic uses. For example, nutriceuticals such as vitamin A (retinol), vitamin D (calciferol), vitamin E, tocopherols, vitamin K (quinone), beta-carotene (pro-vitamin-A) and combinations thereof.

Generally, amounts of therapeutic agent, when used, can be quite variable depending upon the agent or agents used. As such, the concentration of therapeutic agent can be at least about 0.005 w/v %, more typically at least about 0.01 w/v % and even more typically at least about 0.1 w/v %, but typically no greater than about 10 w/v %, more typically no greater than about 4.0 w/v %, still more typically no greater than about 2.0 w/v %.

The emulsions of the present invention may optionally comprise one or more additional excipients and/or one or more additional active ingredients. Excipients potentially used in the ophthalmic emulsions include, but are not limited to, demulcents, tonicity agents, preservatives, chelating agents, buffering agents, and surfactants. Other excipients comprise solubilizing agents, stabilizing agents, comfort-enhancing agents, polymers, emollients, pH-adjusting agents and/or lubricants.

The emulsion is typically aqueous and therefore includes a substantial amount of water, which is typically purified. The emulsion typically includes water at a concentration of at least about 50 w/v %, more typically at least about 85 w/v % and even more typically at least about 93 w/v %, but typically no greater than about 99.99 w/v %, more typically no greater than about 99.0 w/v %, still more typically no greater than about 0.3 w/v % and even still more typically no greater than about 98 w/v %.

The emulsion of the present invention may be formed using a variety of combining and mixing protocol and techniques known to those skilled in the art. According to one preferred embodiment, however, the ingredients are mixed and combined according to a specific protocol. In such protocol, multiple admixtures are formed and those admixtures are combined to form the emulsion. The following lab compounding process was used as starting point:
1. Emulsion Preparation
   Weigh mineral oil and heat to approx. 70° C. Add polyoxyl 40 stearate, sorbitan tri-stearate and other surfactants in mineral oil, mix until completely dissolved.
   Weigh pH adjusted WFI water and heat to approx. 70° C. Add DMPG-Na, mix until completely dissolved.
   Combine aqueous and oil phases and mix until homogenous.
   Homogenize the above homogenous mixture.
   Transfer emulsion to Microfluidizer and process.
2. HP Guar Solution Preparation and Sterilization
   Collect pH adjusted WFI in a depyrogenated glass. While mixing using overhead mixer, add HP guar slowly until the HP guar is fully hydrated and homogeneous.
   Perform polish filtration on the HP Guar solution and transfer an appropriate amount to the final compounding vessel.
   Add appropriate amount of emulsion and mix until homogenous.
   Autoclave HP guar and emulsion mixture at 121° C. for 20 minutes using pre-set liquid cycle.
   Afterwards bring the formulation to room temperature before proceeding to the next step.
3. HA solution preparation and sterilization
   Collect pH adjusted WFI in a depyrogenated glass. While mixing using overhead mixer, add HA slowly until the HA is fully hydrated and homogeneous.
   Filter the solution through a 0.22 μm Sterile filtration unit.
   Transfer and cap the bottle in the Horizontal Laminar Flow Hood.
4. Salt solution preparation and sterilization.
   Collect approx. 10% batch wt. WFI in a depyrogenated glass. Heat to 70° C. and add boric acid, sorbitol, and propylene glycol.
   Stir until dissolved.
   Allow to cool to RT and adjust pH to 6.1 (6.0-6.2) using 6N Sodium Hydroxide.

Sterile filter through 0.22 μm filter and transfer to the laminar flow hood
5. Final Compounding
   Addition of HA and salt solution utilizing aseptic techniques and final compounding.
   Add the HA solution to the HP Guar/Emulsion solution via aseptic pumping.
   Add the salt solution to the formulation via aseptic pumping then mix until homogenous.
   Using IN Sodium Hydroxide or IN Hydrochloric acid, adjust the pH of the formulation to 7 (6.5-7.5).
   QS the formulation to 100% with WFI and mix until homogenous.

Perform bulk formulation visual inspection, to check for any particulates and phase separation.

The emulsion can be used as an ocular lubricant, a drug delivery vehicle or the like. However, it has been found particularly desirable for use as a dry eye therapy. As such, a individual diagnosed with or experiencing dry eye symptoms can dispense the emulsion to that individual's eye for alleviating those dry eye symptoms. Typically the emulsion is provided in an eye dropper such that an individual may instill one, two or more drops into one or both of their eyes on a regular or as needed basis. Upon instillation, the emulsion will typically gel upon the corneal surface of the eye allowing for more significant therapeutic effects such as aiding in the delivery of lipids to the ocular surface.

Advantageously, the stability of the oil in water emulsion of the present invention can facilitate lubrication and/or the delivery of lipids (e.g., lipid therapeutic agents) to the ocular surface. These lipids can aid in stabilizing the tear film and/or can provide alternative therapeutic advantages to the eye. Moreover, the mucoadhesive polymer can aid residence time of the emulsions upon the eye such that the emulsions can be more efficacious.

### Examples

| | CE1 | IE7 | IE8 | IE9 |
|---|---|---|---|---|
| COMPONENT | w/v %. | w/v %. | w/v %. | w/v %. |
| HP-Guar | 0.15 | 0.15 | 0.15 | 0.15 |
| Sodium | 0.15 | 0.15 | 0.15 | 0.15 |
| hyaluronate | | | | |
| Mineral oil | 1.00 | 1.00 | 1.00 | 1.00 |
| Boric Acid | 1.00 | 1.00 | 1.00 | 1.00 |
| Anionic | 0.005 | 0.005 | 0.005 | 0.005 |
| Phospholipid | | | | |
| Polyoxyl 40 | 0.38 | 0.38 | 0 | 0 |
| Stearate (P40) | | | | |
| Sorbitan | 0.29 | 0.29 | 0 | 0 |
| Tristearate (Span 65) | | | | |
| Tween 80 | 0 | 0 | 0.5 | 0 |
| TPGS | 0 | 0.5 | 0.5 | 1.0 |
| Propylene Glycol | 0.6 | 0.6 | 0.6 | 0.6 |
| Sorbitol | 0.7 | 0.7 | 0.7 | 0.7 |
| Sodium Hydroxide | Adjust pH to 7.0 | Adjust pH to 7.0 | Adjust pH to 7.0 | Adjust pH to 7.0 |
| Hydrochloric Acid | Adjust pH to 7.0 | Adjust pH to 7.0 | Adjust pH to 7.0 | Adjust pH to 7.0 |
| Purified Water | QS 100 | QS 100 | QS 100 | QS 100 |
| (HA + HP-Guar)/ | 0.45 | 0.26 | 0.30 | 0.3 |
| (P40 + Span | | | | |
| 65 + TPGS + | | | | |
| Tween 80) | | | | |
| Oil/(P40 + Span | 1.49 | 0.85 | 1.0 | 1.0 |
| 65 + TPGS + | | | | |
| Tween 80) | | | | |
| Transparent | not | yes | yes | yes |
| Particle | 130.4 | 70.5 | 75.6 | 100.2 |
| Diameter(D90), | | | | |
| nm | | | | |

We claim:

1. An antioxidant containing ophthalmic emulsion, the emulsion comprising:
water forming an aqueous phase;
oil forming an oil phase, wherein oil in amount from 0.8 to 1.2 w/v %;
a charged phospholipid;
a first surfactant, wherein the first surfactant is a vitamin-based surfactant (vitamin E TPGS),
a second surfactant, the second surfactant is selected from a group of surfactants consisting of polyoxyethylene sorbitan monooleate (Tween 80), and a hydrophilic surfactant having an HLB value having an HLB value of from 10 to 18, wherein the hydrophilic surfactant does not include vitamin E TPGS and polyoxyethylene sorbitan monooleate, and a hydrophobic surfactant having an HLB value of from 1 to 6, and a combination thereof; borate;
two mucoadhesive polymers, the first mucoadhesive polymer is sodium hyaluronate, the second mucoadhesive polymer is galactomannan polymer;
wherein a ratio of the total concentration of the two mucoadhesive polymers to the total concentration of the first surfactant and the second surfactant is between 0.20 and 0.40;
wherein a ratio oil to the total concentration of the first surfactant and the second surfactant is between 0.7 and 1.1;
wherein the ophthalmic emulsion is transparent; 25
The borate and galactomannan polymer cooperatively act to form a gel upon instillation of the emulsion in an eye of an individual.

2. An emulsion according to claim 1 wherein the oil is a hydrocarbon selected from mineral oil, paraffin oil and petrolatum.

3. An emulsion according to claim 1 wherein the oil is at least 0.9 w/v % and no greater than 1.1 w/v % of the emulsion.

4. An emulsion according to claim 1 wherein the hydrophilic surfactant is present in the emulsion in an amount between 0.15 w/v % to 1.0 w/v %.

5. An emulsion according to claim 1 wherein the hydrophilic surfactant is polyoxyethylene-40-stearate.

6. An emulsion according to claim 1 wherein the hydrophobic surfactant is present in the emulsion in an amount between 0.15 w/v % to 1.0 w/v %.

7. An emulsion according to claim 1 wherein the hydrophobic surfactant is a sorbitan tristearate.

8. An emulsion according to claim 1 wherein the second mucoadhesive galactomannan polymer is selected from the group consisting of guar and hydroxypropyl guar.

9. An emulsion according to claim 1 wherein the second mucoadhesive galactomannan polymer is present in the emulsion at a concentration of at least 0.01 w/v %, but no greater than 1.0 w/v.

10. An emulsion according to claim 1 wherein the first mucoadhesive sodium hyaluronate is present in the emulsion at a concentration of at least 0.01 w/v %, but no greater than 1.0 w/v.

11. An emulsion according to claim 1 wherein the charged phospholipid is an anionic phospholipid named dimyristoyl phosphatidylglycerol.

12. An emulsion according to claim 1 wherein the phospholipid is present in the emulsion in a concentration from 0.05 to 7.0 percent by weight.

13. An emulsion according to claim 1 further comprising a borate/polyol buffer system.

14. An emulsion according to claim 1, further comprising a preservative, wherein the preservative is a polymeric quaternary ammonium compound.

15. An emulsion according to claim 1, wherein the oil phase is in droplets within the aqueous phase and the droplets have an average diameter that is no greater than 120 nm but is at least 50 nm, wherein the average diameter is corresponding to 90% of the cumulative undersize distribution by volume measured using Anton Paar Litesizer 500.
